(19) United States
(12) Patent Application Publication
(10) Pub. No.: US 2003/0031692 A1
(43) Pub. Date: Feb. 13, 2003

(54) Pulverulent cosmetic composition

(76) Inventor: Nathalie Jager Lezer, Verrieres-le-Buisson (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/199,172

(22) Filed: Jul. 22, 2002

(30) Foreign Application Priority Data
Jul. 20, 2001 (FR) 01 09768

(51) Int. Cl. A61K 7/00

(52) U.S. Cl. 424/401

(57) ABSTRACT

A pulverulent cosmetic composition, especially a makeup composition, comprising at least one particulate phase and at least one fatty phase, wherein the composition also comprises a gelling agent of silicone elastomer type with surfactant properties.

The present invention relates to a cosmetic composition, especially a makeup composition, in the form of a powder, for example a free, compact or pressed powder, comprising a gelling agent.

Makeup powders generally comprise, firstly, a particulate phase comprising, for example, pigments and fillers, and secondly, a fatty phase as binder comprising fatty substances, which is intended to give the finished product a certain level of cohesion, to give softness and an emollient property to the makeup product and to promote its adhesion to the skin. They may also comprise a hydrophilic phase, for example an aqueous phase, which gives them freshness properties when applied.

The formulation of binders in powders, such as in compact powders, can raise many difficulties since the final composition should be sufficiently homogeneous and compact to be able to be taken up easily and moreover to prevent fragmentation caused especially by impacts.

Makeup powders are thus products generally comprising a very high content of pulverulent compounds and fatty substances. These products may thus give sensations of tautness or a drying effect when they are applied to the skin.

The matt effect is particularly sought for users with mixed or greasy skin, and also in hot and humid climates. The matt-effect fillers used in makeup powders are usually absorbent fillers, such as those absorbing the sebum and the excess oil provided by the composition, for instance talc, silica, kaolin or nylon powder, or alternatively fillers with light-scattering optical properties, these properties being known under the name “soft-focus” effect. A composition with soft-focus properties is such that, when it is applied to the skin, it gives a blurred effect which hides the microreliefs of the skin. These fillers have a tendency to dry out the skin, to mark out (i.e. to render more visible, accentuate and bring out) the skin relief, such as the wrinkles and pores, thus accentuating local imperfections. Furthermore, absorbent fillers can give the skin an unnatural powdery appearance and have a matt effect that is not particularly long-lasting.

There is thus still a need for a comfortable matt pulverulent composition that marks out less the skin relief, in particular the dry areas of the face.

The inventor has found, unexpectedly, that the use of a gelling agent of silicone elastomer type with surfactant properties in a pulverulent composition makes it possible to obtain a powder that can provide desirable cosmetic and/or optical properties, in terms of giving both a matt effect, and a soft-focus effect.

One aspect of the invention is thus a cosmetic composition in powder form comprising at least one particulate phase and at least one fatty phase, wherein the composition further comprises at lease one gelling agent of silicone elastomer type with surfactant properties and the particulate phase is present in the composition in a content ranging from 50% to 98% by weight relative to the total weight of the composition.

The gelling agent can be dispersed in a liquid fatty phase, for example, a silicone oil or a hydrocarbon oil.

A cosmetic powder with good cohesion enabling ready use for makeup purposes is thus obtained.

The compositions according to the invention can give a matt effect on keratin materials onto which they are applied.

The compositions thus obtained can show excellent dispersion of the pigments. The composition obtained can be very homogeneous and remain so even after application to the skin, for example, for several hours.

The compositions according to the invention can also show excellent cosmetic properties: they can, in at least one embodiment, adhere sufficiently to the skin but not excessively, be very gentle and be easy to apply.

Another aspect of the present invention is a cosmetic makeup or care process for human keratin materials, such as the skin, further such as the skin of the face and the body, comprising the application to these materials of the composition as defined above.

According to the present invention, the expression “keratin materials” means the skin, the nails, the hair, the eyebrows, the eyelashes, mucous membranes (inner edge of the lower eyelids) and semi-mucous membranes (the lips), and any other area of body or facial skin.

Another aspect of the present invention is a method of providing a matt effect to a keratin material, comprising application to the keratin material of the composition according to the present invention.

Another aspect of the present invention is a method of improving the colour in the composition according to the present invention on a keratin material, comprising application to the keratin material of the composition according to the present invention.

Thus, it is possible, by means of one embodiment of the present invention, to produce compositions comprising a smaller amount of pigments than the compositions of the prior art, while at the same time obtaining the same colour intensity. Similarly, by using the same amount of pigments as a composition of the prior art, the use of the gelling agent according to an embodiment of the invention makes it possible to obtain a composition whose colour can be much more vivid, stronger or even more intense, and more developed.

Furthermore, one embodiment of the composition according to the invention is a complexion-correcting composition, i.e. it serves to give the skin or the lips a matt effect and/or to hide imperfections in the skin and/or the lips. The invention thus also relates to a method of providing a matt effect to the skin and/or lips, and/or hiding imperfections of the skin and/or lips, comprising application to the skin and/or lips of the composition according to the present invention. Furthermore, another aspect of the present invention relates to a method of manufacturing a care or makeup composition, with the aim of giving a matt effect to a keratin material, and/or hiding imperfections of the skin and/or lips, comprising including the composition according to the present invention.

The term “imperfections” means any skin defect, for instance the microrelief (pores, wrinkles and fine lines), marks and blackheads, and blood capillaries.

The compositions according to the invention can find an application in one aspect in the field of skin makeup and/or care, such as in the field of makeup products for the face and the body. Consequently, the composition according to the invention can be in the form of an eyeshadow, a face powder, an eyebrow makeup, a face and/or body powder, a concealer product, a matt-effect product or a body makeup product.

Other characteristics, aspects and advantages of the present invention in its various embodiments will become apparent on reading the detailed description that follows.

The compositions according to the invention comprise at least one gelling agent of silicone elastomer type with surfactant properties, which may, for example, represent up to 10% by weight, such as from 0.3% to 10% by weight and further such as from 0.6% to 6% by weight relative to the total weight of the composition.

The term “elastomer” means a supple, deformable material with viscoelastic properties and can have the consistency of a sponge or a supple sphere. Its modulus of elasticity is such that this material can withstand deformation and has a limited capacity for extension and contraction. This material is capable of regaining its original shape after being stretched. This elastomer can be formed from high molecular weight polymer chains whose mobility is limited by a uniform network of crosslinking bridges.

The gelling agent according to the invention can be a crosslinked solid elastomeric organopolysiloxane in the form of particles and comprises at least one oxyalkylenated group, such as an oxyethylenated (OE) group, for example from 1 to 40, such as from 1 to 20, and further such as from 10 to 20 and even further such as from 12 to 18 and even further such as from 12 to 20 oxyalkylenated units, which may form polyoxyalkylene, such as polyoxyethylene chains. For example, the elastomeric organopolysiloxane can comprise only oxyethylenated groups as oxyalkylenated groups. Such groups give the gelling agent according to the invention desired surfactant properties. These groups may be pendent, at the end of a chain and/or intended to link two parts of the silicone structure. The number of silicons bearing these groups can be, for example, from 1 to 10, such as from 1 to 6.

According to the invention, the expression “oxyalkylenated group” means a group comprising from 2 to 4 carbon atoms, such as oxyethylenated and oxypropylenated groups.

Although an exemplary embodiment of the invention relates to gelling agents comprising only oxyethylenated groups as oxyalkylenated groups, other exemplarly embodiments of the invention may relate to gelling agents comprising only oxypropylenated groups as oxyalkylenated groups. The gelling agents may also comprise both at least one oxyethylenated (OE) group, for example 1 to 20, and at least one oxypropylenated (OP) group, for example 0 to 20; these gelling agents are also known as organopolysiloxanes containing alkylethoxy-propylenated group(s). In one embodiment, the number of oxyethylenated groups is greater than the number of oxypropylenated groups.

Moreover, the silicone structure forming the polymer backbone of the gelling agent can, in one embodiment, be a polydimethylsiloxane (PDMS) structure of which optionally a portion of the methyl groups is substituted with C₂ to C₃₀, such as C₈ to C₂₄ and further such as C₁₀ to C₂₀ alkyl groups or phenyl groups, either at the end of a chain or pendent.

Moreover, the organopolysiloxane containing oxyalkylenated group(s) can comprise at least one silicone backbone linked together via at least one oxyalkylenated, such as oxyethylenated group as defined above. For example, it can comprise at least two polymer backbones linked together.

In one embodiment, the at least one silicone backbone of the elastomeric organopolysiloxanes of the composition according to the invention can comprise from 26 to 80 silicon atoms.

According to the invention, the gelling agent may serve as emulsifier for an aqueous phase in a liquid fatty phase.

The gelling agent according to the present invention can thicken a liquid fatty phase and emulsify an aqueous phase in a liquid fatty phase. In one embodiment, the gelling agent does not dry out the skin but provides good cosmetic properties, such as softness and freshness. This elastomeric gelling agent can thus give pulverulent compositions that are comfortable to apply, have a creamy texture, spread on well, are gentle and do not feel sticky. These cosmetic properties are due firstly to the texture of the organopolysiloxane which is the gelling agent, and secondly to their properties that are comparable to those of microsponges trapping the oily media, such as those of the composition and those secreted by the skin.

The property of the compositions according to the invention to give the skin a matt effect is noteworthy, and in some embodiments of the invention is attributed at least partly to the presence of these organopolysiloxanes containing oxyalkylenated, such as oxyethylenated group(s).

According to one embodiment of the invention, the composition of the invention is a simple or multiple emulsion, with a lipophilic continuous phase, such as with a liquid fatty continuous phase, and is free of surfactant other than the gelling agent according to the invention.

The composition according to the invention can be stable, i.e. it does not demix at room temperature for at least 2 months.

In one embodiment of the invention, the gelling agent according to the invention is a partially or totally crosslinked elastomeric organopolysiloxane of three-dimensional structure, present in a liquid fatty phase, such as an oily phase. When included in a liquid fatty phase, such as an oily phase, it becomes converted, depending on the degree of liquid fatty phase used, from a product of spongy appearance when it is used in the presence of small amounts of liquid fatty phase, to a uniform gel, in the presence of larger amounts of liquid fatty phase. The gelling of the liquid fatty phase by this elastomer may be total or partial.

According to such an embodiment, the elastomeric gelling agent of the invention is present in a liquid fatty phase, such as in a dispersed form, and is thus in the form of a paste or a gel.

The expression “liquid fatty phase”, also referred to as an “oily phase”, means any non-aqueous substance or mixture of non-aqueous substances, which is liquid at room temperature (about 25° C.) and atmospheric pressure (about 1.013×10⁵ Pa).

The gelling agent according to the invention may be chosen from crosslinked polymers obtained by addition and crosslinking reaction in non-aqueous medium, in the presence of a catalyst, such as a catalyst of platinum type, of at least:

(a) one first organopolysiloxane (i) comprising at least two vinyl groups in α-ω positions of the silicone chain; and

(b) a second organopolysiloxane (ii) comprising at least one hydrogen atom linked to a silicon atom per molecule and at least one oxyalkylenated, such as oxyethylenated group.

For example, the organopolysiloxane (i) is chosen from polydimethylsiloxanes (PDMS), such as α,ω-dimethylvinylpolydimethylsiloxanes. The organopolysiloxane (ii) is chosen, for example, from polydimethylsiloxanes comprising at least one hydrogen atom, each linked to a silicon atom, and at least one oxyalkylenated, such as oxyethylenated group, and optionally at least one oxypropylenated group, linked to a silicon atom via an alkylene radical comprising from 1 to 22 carbon atoms.

Optionally, the silicone chains of the first and second organopolysiloxanes (i) and (ii) comprise at least one chain chosen from pendent C₁ to C₆ alkyl chains and aryl chains.

As mentioned above, the elastomeric organopolysiloxane may be in a liquid fatty phase with which it forms an anhydrous gel. This gel may be obtained, for example, as follows:

(a) mixing the first organopolysiloxane (i) and the second organopolysiloxane (ii);

(b) adding the liquid fatty phase, which can be oily, to the mixture from step (a); and

(c) polymerizing the first organopolysiloxane (i) and the second organopolysiloxane (ii) in a liquid fatty phase, which can be oily, in the presence of a platinum catalyst.

The oily phase that may be used during the manufacture of the anhydrous gel comprises at least one oil that is liquid at room temperature (25° C.), chosen from hydrocarbon oils and silicone oils. For example, the oily phase can be a silicone liquid phase, comprising at least one oil chosen from linear-chain and cyclic-chain PDMSs that are liquid at room temperature, optionally comprising an alkyl or aryl chain that is pendent or at the end of a chain, the alkyl chain comprising from 1 to 6 carbon atoms.

The expression “hydrocarbon oils” means oils primarily comprising carbon atoms and hydrogen atoms, such as alkyl and alkenyl chains, for instance alkanes and alkenes. These oils may also comprise at least one ester, ether, hydroxyl or carboxylic group.

The silicone elastomer with surfactant properties may, for example, be present in the composition in a content, of active material, ranging from 0.3% to 10% by weight, such as from 0.6% to 6% by weight, relative to the total weight of the composition.

The gelling agent according to the invention is, for example, an organopolysiloxane obtained according to the procedure of Examples 3, 4 and 8 of U.S. Pat. No. 5,412,004 and of the examples of U.S. Pat. No. 5,811,487, the disclosures of both of which are incorporated by reference herein.

The silicone elastomer with surfactant properties of the invention is chosen, for example, from those sold under the names KSG 21, KSG 31, KSG 32, KSG 41 and KSG 50 by the company Shin Etsu, DC 9010 and DC 9011 by the company Dow Corning, or alternatively may be the product of Example 3 of U.S. Pat. No. 5,412,004.

The product of Example 3 of U.S. Pat. No. 5,412,004 is in the form of a pasty gel comprising about 33% by weight of crosslinked organopolysiloxane containing oxyethylenated group(s) and about 67% of PDMS 6 cSt. The organopolysiloxane contains about 18% by weight, relative to the total weight of the polymer, of ethylene oxide.

The gelling agent according to the invention, as a mixture with oil, for example at 33% by weight in PDMS 6 cSt, can have at least one of the following properties:

the gelling agent has plastic shear-thinning behaviour with a dynamic viscosity at low shear in the region of 10⁻³ s⁻¹ or 10⁴ s⁻¹, ranging from 2×10⁶ P to 4×10⁶ P and a dynamic viscosity of 15 to 50 P (1.5 to 5 Pa.s) for a shear rate of 200 s⁻¹ at t=10 min, measured using an RS 75 controlled-stress rheometer (Haake) at 25° C. in cone/plate geometry; cone characteristics: 20 mm diameter, 1° angle and 40 μm gap;

the gelling agent has viscoelastic behaviour at 1 Hz with a dominant elastic nature at low shear stress values defined as follows: 800 PA<G*ₚₗₐₜₑₐᵤ<2,500 Pa, with δₚₗₐₜₑₐᵤ in the region of 10°, G*ₚₗₐₜₑₐᵤ representing the consistency and δₚₗₐₜₑₐᵤ representing the elasticity;

the gelling agent has a flashpoint of about 160 to 170° C. at atmospheric pressure. For the gel of Example 3 of U.S. Pat. No. 5,412,004, the dynamic viscosity is about 45 P (4.5 Pa.s) at a shear rate of 200 s⁻¹.

This gelling agent can be stable at room temperature (25° C.) for at least 4 months (no exudation of oil).

The particles of elastomeric organopolysiloxane (active material) of this gelling agent have a size ranging from 0.1 to 500 μm, such as from 3 to 200 μm and further such as from 3 to 50 μm, as measured by methods known to those skilled in the art. These particles may be spherical, flat or amorphous, such as with a substantially spherical, elliptical or oblong shape.

The gelling agent of the invention can be, for example, a surfactant with an HLB (hydrophilic-lipophilic balance) of about 2.5. It is thus suitable for manufacturing a stable water-in-oil emulsion or a stable oil-in-water-in-oil or water-in-oil-in-water emulsion.

This gelling agent may be combined with a fatty phase comprising fatty substances that are liquid at room temperature, waxes and/or gums that are solid at room temperature, pasty fatty substances of animal, plant, mineral or synthetic origin, and mixtures thereof.

The fatty phase may comprise products that are liquid at room temperature, such as oils, for instance silicone oils, fluoro oils, fluorosilicone oils or hydrocarbon oils that may be partially siliconized. These oils may be volatile at room temperature and atmospheric pressure. The term “volatile oil” means an oil that is liable to evaporate, in less than one hour, on contact with the skin or the lips, for example, having a non-zero vapour pressure, such as ranging from 0.133 to 3.99×10⁴ Pa (at room temperature and atmospheric pressure) and further such as greater than 3.99 Pa.

The fatty phase generally represents, for example, from 1.5% to 49.7% by weight, such as from 1.5% to 40% by weight and further such as from 1.5% to 30% by weight relative to the total weight of the composition.

Examples of oils, which can be used in the composition of the invention, include:

hydrocarbon oils of animal origin, such as perhydrosqualene;

hydrocarbon plant oils such as liquid triglycerides of fatty acids, for example sunflower oil, corn oil, soybean oil, marrow oil, grape pip oil, sesame oil, hazelnut oil, apricot oil, macadamia oil, castor oil, avocado oil, caprylic/capric acid triglycerides such as those sold by the company Stearineries Dubois and those sold under the names Miglyol 810, 812 and 818 by the company Dynamit Nobel;

oils of formula R¹COOR² in which R¹ represents a higher fatty acid residue comprising from 7 to 19 carbon atoms and R² represents a branched hydrocarbon chain comprising from 3 to 20 carbon atoms, such as, purcellin oil, isopropyl myristate and octanoates, decanoates and ricinoleates of alcohols and of polyalcohols;

linear and branched hydrocarbons of mineral and synthetic origin, such as volatile and non-volatile liquid paraffins and derivatives thereof, petroleum jelly, polydecenes and hydrogenated polyisobutene such as parleam;

synthetic ethers of formula R³COR⁴ in which R³ is a C₃ to C₁₉ alkyl radical and R⁴ is a C₃ to C₂₀ alkyl radical;

fatty alcohols such as octyldodecanol or oleyl alcohols;

partially hydrocarbon and/or silicone-containing fluoro oils, such as perfluoropolyesters;

silicone oils such as polymethylsiloxanes comprising a linear or cyclic silicone chain, which are liquid or pasty at room temperature, phenyl dimethicones, phenyl trimethicones and polymethylphenylsiloxanes, and alkylpolydimethylsiloxanes with a C₂ to C₂₀ alkyl chain; and

mixtures thereof.

As an example, the oil used according to the present invention can be a silicone oil.

The gelling agent, which is an organopolysiloxane gel containing oxyethylenated group(s) allows these oils to be structured in the form of a novel texture of “flan” type, free of oily gelling agent which would hinder the soft/silky and pleasant feel of the composition.

The composition according to the invention can further comprise at least one wax and/or one gum chosen from hydrocarbon, fluoro and silicone gums and waxes and mixtures thereof, which may be solid or semi-solid (in the form of a paste) at room temperature. These waxes can be of plant, mineral, animal and/or synthetic origin. These waxes can, for example, have a starting melting point of greater than 25° C., such as greater than 45° C., at atmospheric pressure.

According to the invention, a wax is a lipophilic fatty substance, which is solid at room temperature, can undergo reversible solid/liquid changes of state, can have a starting melting point of up to 200° C. and can have an anisotropic crystal organization in the solid state. By bringing the wax to its melting point, it is possible to make it miscible with the liquid fatty phase and to form a microscopically homogeneous mixture, and then by returning the temperature of the mixture to room temperature, crystallization of the wax in the liquid fatty phase of the mixture can be obtained.

The waxes may be silicone waxes, i.e. waxes comprising a silicone structure and units comprising at least one chain chosen from alkyl and alkoxy chains pendent and/or at the end of a silicone structure, the at least one chain being linear or branched and comprising from 10 to 45 carbon atoms. These waxes are referred to, respectively, as alkyl dimethicones and alkoxy dimethicones. Moreover, the alkyl chain can comprise at least one ester function.

Other examples of the waxes, which can be used in the invention, include waxes of animal origin such as lanolin and beeswax; waxes of plant origin such as carnauba wax and candelilla wax; waxes of mineral origin, for example paraffin wax, lignite wax and microcrystalline waxes, ceresin and ozokerite; and synthetic waxes, such as polyethylene waxes.

These fatty substances can be chosen in a varied manner by a person skilled in the art to prepare a composition having at least one desired property, for example in terms of consistency or texture.

For example, the presence of waxes makes it possible to obtain good mechanical strength, such as when the composition is in the form of a pressed or cast product.

The compositions according to the invention also comprise a particulate phase which may comprise pigments and/or nacres and/or fillers and/or flakes usually used in cosmetic compositions or known to those skilled in the art for fulfilling such a use, and/or mixtures thereof. The term “particulate phase” means a phase comprising components in particulate form, which may be of different sizes and/or of different natures, and may consist almost essentially of such particles. The term “particulate phase” should also be understood as meaning a combination of particles of any form (for example, platelet or lamellar, spherical or oblong form or any similar form), irrespective of their crystallographic form (for example, leaflet, cubic, hexagonal or orthorhombic type).

The particles of the particulate phase may, for example, be coated with at least one silicone compound such as polydimethylsiloxanes and/or with polymers, for example, polyethylenes and/or at least one fluoro compound and/or at least one amino acid.

The term “pigments” should be understood as meaning white or coloured, mineral or organic particles, which are insoluble in the medium, intended to colour and/or opacify the composition.

The pigments may be present, for example, in a content ranging from 0.1% to 30%, such as from 0.5% to 15% by weight relative to the total weight of the particulate phase. They may be white or coloured, mineral and/or organic, and of usual or nanometric size. Among the mineral pigments and nanopigments that may be mentioned are titanium dioxide, zirconium dioxide and cerium dioxide, and also zinc oxide, iron oxide and chromium oxide, nanotitaniums, nanozincs and ferric blue. Among the organic pigments that may be mentioned are carbon black and lakes, for instance salts of calcium, of barium, of aluminium and of zirconium, and acid dyes such as haloacid dyes, azo dyes and anthraquinone dyes.

The pigments may, for example, be coated with silicone compounds such as polydimethylsiloxanes and/or with polymers, such as polyethylenes. Mention may thus be made of the “SI oxides”, which are polymethylhydrogenosiloxane-coated pigments sold by the company Myioshi.

The term “fillers” should be understood as including colourless and white, mineral and synthetic, lamellar and non-lamellar particles intended to give body and/or rigidity to the composition, and/or to give softness, a matt effect and uniformity to the makeup.

The fillers may be present, for example, in the particulate phase in a content ranging from 70% to 100% by weight relative to the total weight of the particulate phase. They may be mineral and synthetic. Mention may be made of talc, mica, silica, kaolin, nylon powder, poly-β-alanine powder and polyethylene powder, Teflon, lauroyllysine, starch, boron nitride, bismuth oxychloride, tetrafluoroethylene polymer powders, polymethyl methacrylate powders, polyurethane powders, polystyrene powders, polyester powders, synthetic hollow microspheres, such as Expancel (Nobel Industrie), microsponges, for instance Polytrap (Dow Corning), sericites, clays and methylsesquioxane resin microbeads (for example Tospearls from the company Toshiba), zinc oxide and titanium oxide, zirconium oxide and cerium oxide, precipitated calcium carbonate, magnesium carbonate and magnesium hydrocarbonate, hydroxyapatite, hollow silica microspheres (Silica Beads from the company Maprecos), glass microcapsules and ceramic microcapsules; metal soaps derived from organic carboxylic acids comprising from 8 to 22 carbon atoms, such as from 12 to 18 carbon atoms per molecule, for example, zinc, magnesium and lithium stearate, zinc laurate and magnesium myristate, and mixtures thereof.

The term “nacres” should be understood as meaning iridescent particles that can reflect light.

Among the nacres that may be envisaged, mention may be made of natural mother-of-pearl, mica coated with any of titanium oxide, iron oxide, aluminium hydroxide, magnesium hydroxide, silica, natural pigment, and bismuth oxychloride, and also coloured titanium mica. The compositions according to the invention may also comprise flakes.

The at least one particulate phase can be present in the compositions according to one embodiment of the invention in a content ranging from 70% to 97% by weight relative to the total weight of the composition.

The composition according to one embodiment of the invention, for example, has the following formulation, on a weight basis relative to the total weight of the composition:

from 0.3% to 10%, such as from 0.6% to 6% of gelling agent of silicone elastomer type with surfactant properties (polymer active material),

from 50% to 98%, such as from 70% to 97% of particulate phase, and

from 1.5% to 49.7%, such as from 1.5% to 40% and further such as from 1.5% to 30% by weight relative to the total weight of the composition, of a fatty phase, which comprises from 0.6% to 40%, such as from 0.6% to 30% and further such as from 0.6% to 20% of a liquid fatty phase, the remainder to 100% by weight of fatty phase being at least one element chosen from pasty, solid and semi-solid fatty substances.

According to another embodiment of the invention, the composition may comprise water. The water may form the aqueous phase of an emulsion prepared from the gelling agent, such as a polyoxyalkylenated organopolysiloxane, with the aim of forming an emulsion comprising a lipophilic continuous phase, such as an oil-based continuous phase.

The composition of the invention may thus comprise a hydrophilic compound chosen from water, solvents that are water-miscible in all proportions, for instance polyols (glycerol, diglycerol and ethylene glycol), C₂ to C₅ lower monoalcohols, acetone and diacetone, and mixtures thereof. For example, the content of hydrophilic compound can represent from 0.1% to 20%, such as from 0.2% to 15% by weight relative to the total weight of the composition.

In such an embodiment, the composition can provide freshness, while at the same time can remain comfortable when applied.

Moreover, it is also possible to prepare a composition according to the invention using as binder an emulsion comprising a hydrophilic continuous phase, such as an aqueous continuous phase. In this case, a hydrophilic surfactant other than the gelling agent, i.e. a surfactant with an HLB value generally of greater than 8, for example in a concentration generally greater than that of the gelling agent, is present.

Further, the composition of the invention may comprise at least one aqueous-phase gelling agent as an additive, i.e. compounds capable of giving the composition a gel appearance and of thickening it. Among the aqueous-phase gelling agents which can be used according to the invention, mention may be made of: water-soluble cellulosic gelling agents, such as hydroxyethylcellulose, methylcellulose, hydroxypropylcellulose, carboxyethylcellulose and carboxymethylcellulose; guar gum; quaternized guar gum; nonionic guar gums comprising C₁-C₆ hydroxyalkyl groups; xanthan gum, carob gum, scleroglucan gum, gellan gum, gellan gum and karaya gum; alginates, maltodextrin, starch and its derivatives, hyaluronic acid and its salts; clays, such as montmorillonites, hectorites and bentones, laponites; polymers comprising a carboxylic group, for instance at least partially neutralized, crosslinked polyacrylic acids such as the “Carbopol” or “Carbomer” products from the company Goodrich (Carbomer 980, for example, neutralized with triethanolamine—abbreviated as TEA); polyglyceryl (meth)acrylate polymers; polyvinylpyrrolidone; polyvinyl alcohol; crosslinked acrylamide polymers and copolymers; crosslinked methacryloyloxyethyltrimethylammonium chloride homopolymers; associative polyurethanes, and mixtures thereof.

According to the invention, the at least one aqueous-phase gelling agent, can, for example, be chosen from xanthan gum, clays (such as bentone and laponite), associative polyurethanes, cellulosic thickeners, such as hydroxyethylcellulose, and at least partially neutralized, crosslinked polyacrylic acids, and mixtures thereof.

The composition of the invention may also comprise any additive usually used in the field under consideration, whether it is water-soluble or not, and stable or unstable outside the composition. Thus, the additive may be chosen from water-soluble and liposoluble colorants, antioxidants, essential oils, preserving agents, cosmetic and dermatological active agents such as active agents that are unstable in an oxidizing medium, hydrophilic and lipophilic active agents, liposoluble polymers, for example, hydrocarbon polymers such as polyalkylenes, fatty-phase gelling agents, solvents, odour absorbers, fragrances, electrolytes, for instance divalent and monovalent mineral salts (NaCl, MgCl₂ or MgSO₄), preserving agents, antioxidants, dyestuffs and mixtures thereof, provided that the additive does not destabilize the active agent present in the composition, namely the gelling agent. Depending on its nature, this additive may be introduced into the liquid fatty phase or into the optional aqueous phase.

This additive may be present in the composition in the amounts usually used, for example, in an amount of from 0% to 20%, such as from 0.01% to 20% and further such as from 0.1% to 10% by weight relative to the total weight of the composition. For the electrolytes, at least 30 to 60 milliosmoles or mosm are usually, but not mandatorily, used.

For example, as active agents that are unstable in an oxidizing medium, mention may be made of vitamins, such as ascorbic acid (vitamin C) and its derivatives, for example, its glycosyl and phosphate derivatives, and its esters, for instance ascorbyl acetate, palmitate and propionate, retinol (vitamin A) and its derivatives, for example, its esters, for instance retinol acetate, palmitate and propionate; urea; rutin; enzymes such as lipase, protease, phospholipase and cellulases; natural extracts such as green tea, extract of balm, extract of thyme, procyannidol oligomers (PCOs) such as hawthorn PCO, pine PCO and grape PCO; certain acids such as kojic acid, caffeic acid, retinoic acid and its derivatives, benzene-1,4-bis(3-methylidene-10-camphorsulphonic acid); carotenoids such as carotenes such as, for example, α-, β- and γ-carotenes, β,φ-carotene, ξ-carotene, β,λ-carotene and lycopene (ψ,ψ-carotene); polyunsaturated fatty acids such as gamma-linolenic acid, and mixtures thereof. The active agent may also be chosen from any natural and synthetic compounds that may comprise the active agents indicated above, such as plant extracts, and further such as fruit extracts.

Needless to say, a person skilled in the art will take care to select the optional additional additives and/or the amount thereof such that at least one advantageous property of the composition according to the invention cannot be, or cannot substantially be, adversely affected by the envisaged addition. For example, these additives must not harm the homogeneity, stability, level of comfort, matt effect or freshness of the composition.

The composition according to the invention is generally in the form of a powder, for example a free, compacted or pressed powder. The final presentation form of the powder as a ready-to-use finished product may be varied, such as a spherical or dome shape. Any presentation geometry falls within the context of the present invention. For example, in the case of a free powder, the binder may represent up to 15% by weight, relative to the total weight of the composition, and for example from 1% to 5% by weight. For a compact powder, the binder content can represent from 1% to 30% by weight, such as from 3% to 20% by weight relative to the total weight of the composition.

The compositions according to the invention may be prepared according to the known methods for preparing cosmetic powders, optionally taking into account the volatility of the oil(s) used, as is known to those skilled in the art.

The examples of compositions below are given as illustrations of the invention without any limiting nature.

### EXAMPLE 1

### According to the Invention

A powder according to the invention was prepared, comprising the constituents expressed below, in weight percentages relative to the total weight of the composition. To this end, the compounds, except for the binder, were mixed together in a Baker Perkins machine. The binder was added with stirring in a Hosokawa Alpine mill.

| | |
|---|---|
| Talc | 83% |
| Polyamide-12 powder | 10% |
| Nanotitanium | 1% |
| Iron oxide pigment | 3% |
| Polyoxyalkylenated silicone | 3% |
| elastomer* | of KSG21 |
| Preserving agent | qs |

*: Of commercial name KSG21 from the company Shin Etsu

### Comparative Example 2

A comparative powder not forming part of the invention was prepared, comprising the constituents below, expressed as weight percentages relative to the total weight of the composition, with the same procedure as in Example 1.

| | |
|---|---|
| Talc | 83% |
| Polyamide-12 powder | 10% |
| Nanotitanium | 1% |
| Iron oxide pigment | 3% |
| Silicone binder | 3% |
| Preserving agent | qs |

The silicone binder is a mixture of the following three constituents:

Abil Wax 9801 from Goldschmidt: polymethylcetyldimethylsiloxane (MW: 900-viscosity 15-25 cSt),

DC Fluid 953 from Dow Corning, mixture of trimethyl siloxysilicate resin and PDMS (33/67, viscosity 700 cSt),

DC Fluid 200 from Dow Corning, PDMS (viscosity 10 cSt).

Assessment

The two compositions of Examples 1 and 2 were assessed by a panel of six consumers, by simultaneous comparison in a half-phase test.

The assessment is established as a function of four criteria, namely:

(1) matt effect at t=0, where t is the time (in contact with the skin, at the time of application)

(2) staying power of the matt effect at t=4 hours

(3) homogeneity of the colour at t=0

(4) staying power of the colour at t=4 hours

An arbitrary graduation scale ranging from 0 to 18.5 was proposed to each consumer, in which 0 is the minimum limit and 18.5 the maximum limit of the property under assessment.

The results are given in the following table.

| | | Powder according | Comparative | |
|---|---|---|---|---|
| | | to the invention | powder of | |
| | | (Example 1) | Example 2 | Difference |
| (1) | Matt effect | 14.5 | 13.1 | 1.4 |
| (2) | Staying power of | 13.6 | 11.7 | 1.9 |
| | the matt effect | | | |
| (3) | Homogeneity of the | 14.6 | 14.3 | 0.3 |
| | colour | | | |
| (4) | Staying power of | 14.0 | 13.7 | 0.3 |
| | the colour | | | |

It is seen that the powder with the greater matt effect (criterion 1) and which is more homogeneous in terms of colour (criterion 3) is that of Example 1 including the silicone elastomer. Four hours after application, it is seen that the powder according to the invention remains more matt than the powder of Comparative Example 2 (criterion 2), with an even greater difference than at the time of application (criterion 1). Furthermore, in terms of homogeneity of the colour, it is seen that the result is just as desirable for the powder according to the invention at t=4 hours (criterion 4) as at the time of application (criterion 3) as for the comparative powder.

What is claimed is:

1. A pulverulent cosmetic composition comprising at least one particulate phase and at least one fatty phase, wherein the composition further comprises at least one gelling agent of silicone elastomer type with surfactant properties and the particulate phase is present in the composition in a content of from 50% to 98% by weight relative to the total weight of the composition.

2. A composition according to claim 1, wherein the at least one gelling agent is chosen from crosslinked solid elastomeric organopolysiloxanes in the form of particles, said elastomeric organopolysiloxanes comprising at least one oxyalkylenated group comprising from 2 to 4 carbon atoms.

3. A composition according to claim 2, wherein the at least one oxyalkylenated group is an oxyethylenated group.

4. A composition according to claim 2, wherein the elastomeric organopolysiloxane are chosen only from oxyethylenated groups .

5. A composition according to claim 2, wherein the elastomeric organopolysiloxane is obtained by addition and crosslinking reaction in non-aqueous medium, in the presence of a catalyst of platinum type, of at least:
one first organopolysiloxane (i) comprising at least two vinyl groups in α-ω positions of the silicone chain per molecule; and
one second organopolysiloxane (ii) comprising at least one hydrogen atom linked to a silicon atom per molecule and at least one oxyalkylenated group comprising from 2 to 4 carbon atoms.

6. A composition according to claim 5, wherein the first organopolysiloxane (i) is chosen from polydimethylsiloxanes.

7. A composition according to claim 6, wherein the first organopolysiloxane (i) is an α,ω-dimethylvinylpolydimethylsiloxane.

8. A composition according to claim 5, wherein the second organopolysiloxane (ii) is chosen from polydimethylsiloxanes comprising at least one hydrogen atom and at least one oxyalkylenated group linked to a silicon atom via an alkylene radical comprising from 1 to 22 carbon atoms.

9. A composition according to claim 5, wherein the at least one oxyalkylenated group is an oxyethylenated group.

10. A composition according to claim 2, wherein the elastomeric organopolysiloxane in the form of particles has a size ranging from 0.1 to 500 μm.

11. A composition according to claim 10, wherein the elastomeric organopolysiloxane in the form of particles has a size ranging from 3 to 200 μm.

12. A composition according to claim 11, wherein the elastomeric organopolysiloxane in the form of particles has a size ranging from 3 to 50 μm.

13. A composition according to claim 1, wherein the at least one fatty phase comprises at least one oil.

14. A composition according to claim 13, wherein the at least one oil is chosen from silicone oils.

15. A composition according to claim 13, wherein the at least one oil is chosen from volatile oils.

16. A composition according to claim 1, wherein the at least one particulate phase is in a content of from 70% to 97% by weight relative to the total weight of the composition.

17. A composition according to claim 1, wherein the at least one gelling agent is in a content of from 0.3% to 10% by weight relative to the total weight of the composition.

18. A composition according to claim 17, wherein the at least one gelling agent is in a content of from 0.6% to 6% by weight relative to the total weight of the composition.

19. A composition according to claim 1, wherein the at least one fatty phase is in a content of from 1.5% to 49.7% by weight relative to the total weight of the composition.

20. A composition according to claim 19, wherein the at least one fatty phase is in a content of from 1.5% to 40% by weight relative to the total weight of the composition.

21. A composition according to claim 20, wherein the at least one fatty phase is in a content of from 1.5% to 30% by weight relative to the total weight of the composition.

22. A composition according to claim 1, wherein the at least one particulate phase comprises at least one component chosen from pigments, nacres, fillers, and flakes.

23. A composition according to claim 22, wherein the pigments are chosen from titanium dioxide, zirconium dioxide and cerium dioxide, zinc oxide, iron oxide and chromium oxide, nanotitaniums, nanozincs, ferric blue, carbon black, salts of calcium, of barium, of aluminium and of zirconium, haloacid dyes, azo dyes and anthraquinone dyes, pigments coated with silicone compounds, and pigments coated with polymers.

24. A composition according to claim 23, wherein the pigments coated with silicone compounds are characterized in that the silicone compounds are chosen from polydimethylsiloxanes.

25. A composition according to claim 22, wherein the pigments are present in a content ranging from 0.1% to 30% by weight relative to the total weight of the particulate phase.

26. A composition according to claim 25, wherein the pigments are present in a content ranging from 0.5% to 15% by weight relative to the total weight of the particulate phase.

27. A composition according to claim 22, wherein the fillers are chosen from talc, mica, silica, kaolin, nylon powders, poly-β-alanine powders and polyethylene powders, lauroyllysine, starch, boron nitride, bismuth oxychloride, tetrafluoroethylene polymer powders, polymethyl methacrylate powders, polyurethane powders, polystyrene powders, polyester powders, synthetic hollow microspheres, microsponges, methylsesquioxane resin microbeads, zinc oxide and titanium oxide, zirconium oxide and cerium oxide, precipitated calcium carbonate, magnesium carbonate and magnesium hydrocarbonate, hydroxyapatite, hollow silica microspheres, glass microcapsules and ceramic microcapsules, and metal soaps derived from organic carboxylic acids comprising from 8 to 22 carbon atoms per molecule.

28. A composition according to claim 27, wherein the metal soaps derived from organic carboxylic acids are characterized in that the organic carboxylic acids comprise from 12 to 18 carbon atoms per molecule.

29. A composition according to claim 22, wherein the fillers are present in a content ranging from 70% to 100% by weight relative to the total weight of the particulate phase.

30. A composition according to claim 22, wherein the nacres are chosen from natural mother-of-pearl, mica coated with titanium oxide, with iron oxide, with aluminium hydroxide, with magnesium hydroxide, with silica, with natural pigment and with bismuth oxychloride, and coloured titanium mica.

31. A composition according to claim 1, comprising, on a weight basis relative to the total weight of the composition:
from 0.3% to 10% of the at least one gelling agent of silicone elastomer type with surfactant properties,
from 50% to 98% of the at least one particulate phase, and
from 1.5% to 49.7% of the at least one fatty phase.

32. A composition according to claim 31, wherein the at least one gelling agent is in a content of from 0.6% to 6% by weight relative to the total weight of the composition.

33. A composition according to claim 31, wherein the at least one particulate phase is in a content of from 70% to 97% by weight relative to the total weight of the composition.

34. A composition according to claim 31, wherein the at least one fatty phase is in a content of from 1.5% to 40% by weight relative to the total weight of the composition.

35. A composition according to claim 34, wherein the at least one fatty phase is in a content of from 1.5% to 30% by weight relative to the total weight of the composition.

36. A composition according to claim 31, wherein the at least one fatty phase comprises from 0.6% to 40% of a liquid fatty phase, the remainder to 100% by weight of the fatty phase being at least one element chosen from pasty, solid and semi-solid fatty substances.

37. A composition according to claim 36, wherein the at least one fatty phase comprises from 0.6% to 30% of a liquid fatty phase.

38. A composition according to claim 37, wherein the at least one fatty phase comprises from 0.6% to 20% of a liquid fatty phase.

39. A composition according to claim 1, further comprising a hydrophilic compound chosen from water and water-miscible solvents.

40. A composition according to claim 39, wherein the hydrophilic compound is in a content of from 0.1% to 20% by weight relative to the total weight of the composition.

41. A composition according to claim 40, wherein the hydrophilic compound is in a content of from 0.2% to 15% by weight relative to the total weight of the composition.

42. A composition according to claim 39, further comprising at least one aqueous-phase gelling agent chosen from hydroxyethylcellulose, methylcellulose, hydroxypropylcellulose, carboxyethylcellulose and carboxymethylcellulose, guar gum, quaternized guar gum, nonionic guar gums comprising C₁-C₆ hydroxyalkyl groups, xanthan gum, carob gum, scleroglucan gum, gellan gum and karaya gum, alginates, maltodextrin, starch and its derivatives, hyaluronic acid and its salts, montmorillonites, hectorites, and bentones, laponites, at least partially neutralized, crosslinked polyacrylic acids, polyglyceryl (meth)acrylate polymers, polyvinylpyrrolidone, polyvinyl alcohol, crosslinked acrylamide polymers and copolymers, crosslinked methacryloyloxyethyltrimethylammonium chloride homopolymers, and associative polyurethanes.

43. A composition according to claim 1, further comprising an additive chosen from water-soluble and liposoluble colorants, antioxidants, essential oils, preserving agents, cosmetic and dermatological active agents, polyalkylenes, fatty-phase gelling agents, solvents, odour absorbers, fragrances, divalent and monovalent mineral salts, preserving agents, antioxidants and dyestuffs.

44. A composition according to claim 1, characterized in that the composition is a makeup composition.

45. A composition according to claim 1, characterized in that the composition is in the form of an eyeshadow, a face powder, an eyebrow makeup, a face and/or body powder, a concealer product, a matt-effect product or a body makeup product.

46. A cosmetic makeup or care process for a human keratin material, comprising applying to the keratin material an effective amount of a composition, comprising at least one particulate phase and at least one fatty phase, wherein the composition further comprises at least one gelling agent of silicone elastomer type with surfactant properties and the particulate phase is present in the composition in a content of from 50% to 98% by weight relative to the total weight of the composition.

47. A cosmetic makeup or care process according to claim 46, wherein the human keratin material is chosen from facial and body skin.

48. A method of providing a matt effect to a keratin material, comprising applying to the keratin material an effective amount of a pulverulent cosmetic composition, comprising at least one particulate phase and at least one fatty phase, wherein the composition further comprises at least one gelling agent of silicone elastomer type with surfactant properties and the particulate phase is present in the composition in a content of from 50% to 98% by weight relative to the total weight of the composition.

49. A pulverulent cosmetic composition for providing a matt effect to a keratin material, wherein the composition comprises at least one particulate phase and at least one fatty phase, wherein the composition further comprises at least one gelling agent of silicone elastomer type with surfactant properties and the particulate phase is present in the composition in a content of from 50% to 98% by weight relative to the total weight of the composition.

50. A method of improving the development of the colour in a pulverulent cosmetic composition on a keratin material, comprising applying to the keratin material an effective amount of a composition comprising at least one particulate phase and at least one fatty phase, wherein the composition further comprises at least one gelling agent of silicone elastomer type with surfactant properties and the particulate phase is present in the composition in a content of from 50% to 98% by weight relative to the total weight of the composition.

51. A pulverulent cosmetic composition for improving the development of the colour in the composition on a keratin material, wherein the composition comprises at least one particulate phase and at least one fatty phase, wherein the composition further comprises at least one gelling agent of silicone elastomer type with surfactant properties and the particulate phase is present in the composition in a content of from 50% to 98% by weight relative to the total weight of the composition.

52. A method of manufacturing a care or makeup composition, comprising including in the composition at least one particulate phase and at least one fatty phase, wherein the composition further comprises at least one gelling agent of silicone elastomer type with surfactant properties and the particulate phase is present in the composition in a content of from 50% to 98% by weight relative to the total weight of the composition.

53. A method of hiding imperfections of skin and/or lips, comprising applying to the skin and/or the lips an effective amount of a composition comprising at least one particulate phase and at least one fatty phase, wherein the composition further comprises at least one gelling agent of silicone elastomer type with surfactant properties and the particulate phase is present in the composition in a content of from 50% to 98% by weight relative to the total weight of the composition.

54. A method of providing a matt effect on skin and/or lips, comprising applying to the skin and/or the lips an effective amount of a composition comprising at least one particulate phase and at least one fatty phase, wherein the composition further comprises at least one gelling agent of silicone elastomer type with surfactant properties and the particulate phase is present in the composition in a content of from 50% to 98% by weight relative to the total weight of the composition.

55. A composition for hiding imperfections of skin and/or lips, wherein the composition comprises at least one particulate phase and at least one fatty phase, wherein the composition further comprises at least one gelling agent of silicone elastomer type with surfactant properties and the particulate phase being is in the composition in a content of from 50% to 98% by weight relative to the total weight of the composition.

56. A composition for providing a matt effect on skin and/or lips, wherein the composition comprises at least one particulate phase and at least one fatty phase, wherein the composition further comprises at least one gelling agent of silicone elastomer type with surfactant properties and the particulate phase is present in the composition in a content of from 50% to 98% by weight relative to the total weight of the composition.

57. A composition according to claim 39, further comprising at least one aqueous-phase gelling agent chosen from xanthan gum, bentonite, laponite, associative polyurethanes, hydroxyethylcelluloses, and at least partially neutrialized, cross-linked polyacrylic acids.
